(19) United States
(12) Patent Application Publication
(10) Pub. No.: US 2020/0410891 A1
(43) Pub. Date: Dec. 31, 2020

(54) COMPUTER SYSTEMS AND METHODS FOR CREATING AND MODIFYING A MULTI-SENSORY EXPERIENCE TO IMPROVE HEALTH OR PERFORMRANCE

(71) Applicant: COGNIFISENSE, INC., Sunnyvale, CA (US)

(72) Inventors: Tassilo BAEUERLE, Sunnyvale, CA (US); Harald F. STOCK, Sunnyvale, CA (US)

(21) Appl. No.: 16/980,937

(22) PCT Filed: Mar. 19, 2019
(86) PCT No.: PCT/US2019/023018

§ 371 Date: Sep. 15, 2020

Related U.S. Application Data
(60) Provisional application No. 62/644,798, filed on Mar. 19, 2018.

(51) Int. Cl. G09B 19/00; G06T 17/00; G09B 9/00; G16H 20/70

(52) U.S. Cl. CPC G09B 19/00; G16H 20/70; G09B 9/00; G06T 17/00

(57) ABSTRACT

Computer systems and methods can include generating a multidimensional sensory environment using an immersive technology, creating a first digital model that includes a visual representation of an emotional, psychological, or somatosensory user experience or aspect of the user experience, receiving a description of an extra-visual sensory signal, layering the extra-visual sensory signal onto the first digital model such that the extra-visual sensory signal is configured to be produced by a sensory device, and producing a corporealized form of the user experience or aspect of the user experience in the multidimensional sensory environment by at least displaying the visual representation of the first digital model in the multidimensional sensory environment via the immersive technology and producing the extra-visual sensory signal associated with the first digital model at the sensory device. The corporealized user experience can be affected to increase user health and/or performance.

### BACKGROUND

Humans have difficulty managing, affecting or even understanding a multitude of psychological experiences or the psychological aspects associated with many physical experiences. For example, emotions are an integral part of human life. They can enrich or deplete life, and they can, at times, be challenging to manage or understand. Emotions, like many psychological experiences, are associated with the many and varied aspects of life, health, and human performance. Almost every physical experience or mental experience or state can be associated with an emotional or psychological component. In some cases, the experience can include a somatosensory component where a physical and/or emotional “sensation” is “felt”, but which may be difficult to pinpoint, localize, or describe; such somatosensory components may also sometimes be understood and communicated in colloquial terms; e.g., “I feel butterflies in my tummy.”

In general, it is difficult for individuals to separate or manage the complex emotional and psychological components of an experience. For example, people often have difficulty disentangling emotion and psychological experiences and states. They also are often unable to separate a plethora of different emotions or psychological experiences from facts—separating reality from their emotions about reality. In some instances, a person may not know why they are experiencing an emotion. There are even conditions, such as alexithymia, that makes it difficult to recognize or describe emotions, which, in turn, may negatively influence behavior.

Moreover, the emotional and/or psychological component of a physical experience can become disconnected from an actual physical experience. For example, a person long suffering from pain might face overwhelming, uncontrolled negative emotions based on many months of pain, even if the source of the pain has healed. This can lead to catastrophizing and chronification of the pain itself. In another example, an endurance athlete may struggle with managing the emotions and physiological feedback associated with the physical challenge, even if the person's body is fully capable of performing.

Complex psychological and emotional experiences are difficult to visualize or communicate to others. This difficulty can be compounded when these psychological and emotional experiences are associated with a physiological experience. As a result, many healthcare practitioners are set with the monumental task of interpreting and treating conditions that involve complex, intertwined physical and psychological components, which they—and the patient—may not fully pinpoint or understand, resulting in less optimal and/or incomplete treatments. Current systems and methods fall short of providing individuals with a medium to accurately or completely express and/or visualize their experience, and there are no systems currently available for effectively communicating the user's experience to healthcare providers in a manner that allows for effective, personalized therapies. Further, current systems fail to address the need in the industry for technologies that can positively affect, heal, or otherwise treat emotional and psychological components of an individual's experience. Therefore, there is a need for an effective way to visualize, communicate, and/or treat complex emotional, psychological, and/or physiological experiences.

### BRIEF SUMMARY

Embodiments described herein are directed to computer systems and computer-implemented methods for corporealizing and/or affecting a user experience or aspects of a user experience. An exemplary computer system can include one or more processors and one or more hardware storage devices having stored thereon computer-executable instructions that, when executed by the one or more processors, configure the computer system to perform at least the following: (i) generate, via an immersive technology coupled to the computer system, a multidimensional sensory environment; (ii) create a first digital model in the multidimensional sensory environment that comprises a visual representation of an emotional, psychological, or somatosensory user experience or aspect of a user experience; (iii) receive a description of an extra-visual sensory signal, the extra-visual sensory signal being associated with one or more of an aural, haptic, thermal, olfactory, or gustatory signal associated with the first digital model; (iv) layer the extra-visual sensory signal onto the first digital model such that the extra-visual sensory signal is configured to be produced by a sensory device associated with the computer system; and (v) produce a corporealized form of the user experience or aspect of the user experience, wherein producing the corporealized form of the user experience or aspect of the user experience comprises: displaying the visual representation of the first digital model in the multidimensional sensory environment via the immersive technology; and producing the extra-visual sensory signal associated with the first digital model at the sensory device.

The computer-executable instructions of the disclosed computer systems can additionally configure the computer system to instantiate a guided protocol comprising audio-visual, or other multimedia or multi-sensory guidance, to affect a change to the user experience, which can include, for example, reinforcing a user's sense of empowerment to control the user experience, reframing the meaning of one or more aspects of the user experience, and/or identify one or more aspects of the user experience associated with a presence, progression of or impending change in the user experience or behavior.

Embodiments of the present disclosure additionally include computer systems having one or more processors and one or more hardware storage devices having stored thereon computer-executable instructions that, when executed by the one or more processors, configure the computer systems to perform at least the following: (i) generate, via a display technology coupled to the computer system, a multidimensional sensory environment; (ii) create a first digital model in the multidimensional sensory environment that comprises a visual representation of an emotional, psychological, or somatosensory user experience or aspect of a user experience; (iii) produce a corporealized form of the user experience, wherein producing the corporealized form of the user experience comprises displaying the visual representation of the first digital model in the multidimensional sensory environment via the display technology; (iv) create a second digital model in the multidimensional sensory environment that comprises an updated visual representation of the first digital model; and (v) produce a second corporealized form of the user experience, wherein producing the second corporealized form of the user experience comprises displaying the updated visual representation of the second digital model in the multidimensional sensory environment via the display technology.

Computer-implemented methods and computer-program product are additionally disclosed Similar to the systems disclosed herein, the disclosed methods and computer program products can be implemented to corporealize a user experience or aspect of a user experience and/or to affect the corporealized user experience or aspect of the user experience.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be apparent to one of ordinary skill in the art from the description or may be learned by the practice of the teachings herein. Features and advantages of embodiments described herein may be realized and obtained by means of the instruments and combinations particularly pointed out in the appended claims. Features of the embodiments described herein will become more fully apparent from the following description and appended claims.

### BRIEF DESCRIPTION OF THE DRAWINGS

To further clarify the above and other features of the embodiments described herein, a more particular description will be rendered by reference to the appended drawings. It is appreciated that these drawings depict only examples of the embodiments described herein and are therefore not to be considered limiting of its scope. The embodiments will be described and explained with additional specificity and detail through the use of the accompanying drawings in which:

FIG. 1 illustrates an exemplary method for corporealizing one or more aspects of a user experience.

FIG. 2 illustrates an exemplary computer architecture in which embodiments described herein may operate including generating a multidimensional sensory environment to corporealize and affect a user experience.

FIG. 3 illustrates another exemplary method for corporealizing and affecting one or more aspects of a user experience.

FIG. 4 illustrates yet another exemplary method for corporealizing and affecting one or more aspects of a user experience.

FIG. 5 illustrates still another exemplary method for corporealizing and affecting one or more aspects of a user experience.

### DETAILED DESCRIPTION

As discussed above, humans have difficulty managing or affecting a multitude of emotional and psychological experiences. This difficulty also pertains to complex experiences involving emotional and/or psychological aspects of a physical experience. Many human experiences are not distinctly physical or psychological; most have both components. Each can be a reflection or embodiment of the other. Each can definitely influence the other. Individuals often have difficulty comprehending and positively affecting these experiences, at least in part, because of their amorphous, intangible nature and inherent subjectivity that is difficult to effectively communicate.

Instead, people are more adept at influencing, controlling, or exacting dominion over things that are concrete and defined; e.g., things that have a definite “shape” or structure. In particular, people often can deal more easily with things that are corporealized. As used herein, the term “corporealized,” or similar, is intended to include those things that can be defined or represented by a sensory signal in such a way that is creates something with a more defined structure or image, or something which can viewed or experienced by an individual but as distinct or separate from the individual, even if (as may sometimes be the case) the distinction between the two experiences may merge. A corporealized emotion, psychological state, or complex experiences combining at least one of the foregoing with a physiological and/or somatosensory sensation can be represented by sensory signals associated with one, or preferably more than one, of the five senses—sight, sound, touch, smell, and taste—or otherwise identifiable by a human (e.g., somatosensory signals, temperature changes, etc.).

However, current systems and methods for understanding or communicating an individual's emotional, psychological, or complex experience fall short and fail to provide any means for effectively corporealizing or communicating these experiences. Current systems attempt to identify and sometimes quantify complex psychological states or emotions but fail to achieve a solution for corporealizing an individual's dynamic experience(s) so that it can be communicated and/or affected. For example, a variety of standardized psychological questionnaires try to assess, for example, an individual's fear, level of anxiety, or depression. Other approaches ask the individual to correlate their experience using a list of descriptive terms. Still another method asks the individual to describe subcomponents (e.g., valences) of an emotion in an attempt to identify a cognizable psychological state or emotion.

There are significant disadvantages with each of the foregoing approaches. For instance, psychological states or emotions are complex, often ambiguous, and even transient, making it difficult for current systems to adequately communicate or affect an individual's experience. Complicating the use of current approaches to capture or communicate an individual's emotional, psychological, or complex experience is the individual's difficulty in understanding their own emotions or their psychological, physiological, and somatosensory perceptions—in addition to their inability to tease each apart from the other in complex experiences. In short, the individuals often struggle to bring abstract “feelings” (psychological, physiological, and somatosensory perceptions) into a concrete state where they can describe them, much less affect them. There remains a need in the art for computer systems and methods that can corporealize and empower an individual to affect their experience.

At the same time, emotions and many psychological states often can be related to or may even cause a physiological impact. For example, fear is often related to elevated heartrate, increased perspiration and faster breathing. The emotions of fear can sometimes cause the autonomic nervous system to initiate these changes in the body even if the cause of the fear is only imagined. In general, these physical or somatosensory sensations can begin to represent the emotion or psychological state—or aspects of the same. For example, “I feel butterflies in my tummy,” or “Something is wrong. I don't know what. I just have this uneasy feeling in my gut.” Even complex psychological states such as cravings can have somatosensory representations. For example, cravings for sweets, tobacco, or alcohol are not an abstract logical drive (e.g. “My logic tells me it is time for some chocolate”); they include somatosensory components. Current systems generally fail to account for the complex interplay of somatosensory sensations with emotions and/or psychological states. Thus, identifying and including representations of such somatosensory components, along with representations of emotion and/or psychological states, could beneficially lead to powerful therapies for improving health and performance.

Learning paradigms provide a tremendous opportunity for helping individuals change (i.e., to learn skills that allow them to cope better with aspects of their experience). If part of the individual's suffering is related to learned or conditioned changes, it is possible to make further changes toward a more preferable goal by utilizing the principles of (implicit or explicit) learning within the systems disclosed herein. In some embodiments, the systems and methods disclosed herein may enable users to learn healthy coping mechanisms to treat aspects of their emotional, psychological, or complex experiences and advantageously do so in a more efficient manner than with other self- or guided-treatment options previously available owing to the immersive, personalized nature of the disclosed systems.

In a broad sense, embodiments of the present disclosure take emotional, psychological, somatosensory, or physiological experiences, create digital representations of them, and then enable the digital representations to be digitally affected to teach a person to make their own changes to these experiences, or aspects of these experiences—and consequently improve their health and/or performance. Disclosed embodiments enable users to address aspects of their experience(s) separately (or separate but conjointly), and thereby address, realign, or optimize (e.g., for performance) the experience for their benefit. As described in more detail herein, some embodiments enable the corporealization of emotional, psychological, physiological, and/or somatosensory experiences using visually descriptive and/or immersive technologies (e.g., virtual, augmented, or mixed realities or holography) in combination with a sensory device or technology (i.e., devices that engage non-visual senses such as hearing, touch, smell, and taste; or other sensations detectable by the body, such as temperature) in an effort to capture (and communicate) the subject's individual perception of their state/dynamic experience(s). In some instances, the foregoing can be utilized in extended reality neuropsychological training (XRNT) to provide self-help or guided-help to affect one or more aspects of the experience, prevent the progression of the experience, prevent the onset of additional/later aspects or consequences of the experience, or improve performance.

It should be appreciated that when used herein, the terms “extended reality neuropsychological training,” “XRNT,” or similar are intended to encompass the combination of an immersive technology with a sensory device to corporealize a user experience, or aspects of a user experience, and/or to affect a change in the user's experience or aspects of the user's experience. In some embodiments, forms of XRNT can be implemented on visual displays accompanied by a sensory device or technology.

The disclosed systems and methods, particularly those incorporating XRNT, can beneficially enable a richer understanding of each patient's emotional, psychological, physiological, and/or somatosensory experience, facilitate the improved communication of critical diagnostic information between, for example, patients and healthcare personnel, and allow for the tailoring and implementation of patient-specific therapeutic regimens—and can do so in a low cost and repeatable manner. Embodiments can further beneficially enable the identification and mitigation of triggers that cause the onset or exaggeration of an individual's experience or otherwise perpetuate the experience or aspects of the experience (e.g., the cascading process during the onset of a migraine headache episode and/or menopausal symptoms).

As used herein, the term “immersive technology” is intended to include computer-implemented realities, such as augmented reality, virtual reality, mixed reality, and holography. For example, augmented reality (AR) is a live, direct or indirect view of a physical, real-world environment whose elements are augmented (or supplemented) by computer-generated sensory input such as video, animations, graphics, or similar. Augmented reality utilizes a user's existing reality and adds to it via a computing device, display, or projector of some sort. For example, many mobile electronic devices, such as smartphones and tablets, can overlay digital content into the user's immediate environment through use of the device's camera feed and associated viewer. Thus, for example, a user could view the user's real-world environment through the display of a mobile electronic device while virtual objects are also being displayed on the display, thereby giving the user the sensation of having virtual objects integrated into a real-world environment. Custom AR-enabled headsets or other devices can also be used.

Virtual reality (VR) is another example of an immersive technology. In general, VR refers to computer technologies that use virtual reality headsets and/or other peripheral devices to generate three-dimensional environments in which a user can create or interact with virtual images, objects, scenes, places, or characters—any of which can represent real-world or imaginary things. Virtual reality immerses a user in a visually virtual experience and allows the user to interact with the virtual environment. As used herein, the term “virtual reality” or “VR” is intended to include those computer-implemented realities that engage at least the user's sense of sight and that do not display the user's (immediate) surrounding real-world environment.

Another example of an immersive technology is a hybrid reality called mixed reality (MR). Mixed reality represents the merging of real and virtual worlds to produce new environments and visualizations where physical and digital objects co-exist and interact in real time. Many MR implementations place new imagery within a real space and often do so in such a way that the new imagery can interact—to an extent—with what is real in the physical world. For example, in the context of MR, a user may view a white board through an MR-enabled headset and use a digitally-produced pen (or even a capped physical pen) to write on the white board. In the physical world, no writing appears on the white board, but within the MR environment, the user's interaction with a real-world object caused a digital representation of the writing to appear on the white board. In MR systems, some synthetic content can react and/or interact with the real-world content in real time.

Holography is another form of immersive technology compatible with disclosed embodiments of XRNT. A hologram is typically a photographic projection of a light field that appears to be three dimensional and which can be seen with the naked eye.

An umbrella term, namely extended reality (XR), incorporates each of the forms of immersive technology—AR, VR, MR, and holography. As used herein, the term “extended reality” or “XR” refers generally to all real and virtual combined environments and human-machine interactions generated by computer technology or wearables. Extended reality includes all its descriptive forms, such as digital representations made or displayed within AR, VR, MR, or holography.

Accordingly, the immersive technology feature of XRNT provides a visual display of the user's experience. It should be appreciated that “visual displays” or “displays” include devices that provide visual stimuli in the form of images, video, projections, holograms, or the like. Accordingly, a display can include a monitor or screen configured to produce images and/or video. A display can additionally include projectors configured to project images or video onto a surface and those configured for holography. A display can additionally include headsets or eyewear configured for virtual reality, augmented reality, and/or mixed reality. Accordingly, visual aspects of the user's experience can be implemented using a 3D display that provides visual representations on an XR headset or otherwise projects visual representations in an interactive three-dimensional space. Additionally, the visual aspects of the user's experience can be implemented using a 2D display that provides visual representations on a flat display, such as a laptop or desktop monitor, the screen of a mobile electronic device, or similar.

In addition to the immersive technology, XRNT utilizes one or more sensory devices for corporealizing the user's experience. As used herein, the term “sensory device” is intended to include devices that provide any of auditory, tactile, thermal, olfactory, and/or gustatory signals to the individual, which may be related to the individual's experience(s) and/or the information visualized in the display or immersive technology.

While previously alluded to, XRNT can additionally include a training feature that allows an individual to affect one or more aspects of their experience. For example, an aspect of a user's experience can be affected by XRNT by allowing the user to confront and/or exert dominion over a corporeal representation of this aspect, allowing the user to see the experience in a new light and to remove or reduce its effect on the user.

Additionally, or alternatively, the training feature of XRNT can affect aspects of a user's experience by, for example, remediating the effect of the experience. This can include reducing the size or intensity of visual (or other sensory stimuli) associated with aspects of the user's experience, as described herein.

The training feature of XRNT can additionally, or alternatively, affect aspects of a user's experience by, for example, allowing the user to identify—and in some instances interrupt—warning signs, cues, or triggers associated with an experience, as described herein.

The training feature of XRNT can additionally, or alternatively, be used to increase a user's performance by, for example, simulating a user experience related to performance and allowing the user to learn how to cope with the user experience related to performance or reduce the impact of the user experience related to performance during live action (e.g., athletes “hitting the wall” or students' performance in standardized test scenarios) or to enter and remain in a higher-level user experience related to performance (e.g., a focused state or an athlete being in “the zone”) for longer periods of time, as described herein.

In general, the corporealization of the user's experiences via XRNT makes these experiences, at least to the user, “real” or tangible. That is to say, embodiments of the present disclosure allow a user to give “physical form” to different aspects of their experience—and in a way that reflects how the specific user actually perceives each aspect of their experience.

By doing so, XRNT addresses one or more problems in the art by providing a medium by which an individual's emotional, psychological, or complex experiences can be corporealized (e.g., experienced through sight and other senses like touch, hearing, smell, and taste) and affected. The corporealization can be shared with another person, including a healthcare provider, who can visualize (e.g., visually and in some embodiments with at least one additional sensory stimulus) the individual's experience as that individual perceives their own experience. A better-informed conversation, diagnosis, and/or treatment can be had with the enhanced information provided by embodiments of XRNT technologies—and with far richer and more concrete information than previous systems and methods in the art.

As provided above, the effectiveness of corporealizing a user's experience or aspects of the user's experience can rely on corporealization via the immersive technology in addition to stimulating one or more extra-visual senses using a sensory device. For example, tactile signals, such as a vibrations, throbs, or pokes, can be provided through a wearable that houses a haptic element (e.g., haptic clothing like a haptic vest, haptic suit, and/or haptic gloves or a handheld device having a resonant actuator or the like). Such a device can be used to augment the power or illusion of the experience (e.g., a physiological and/or psychological aspect of pain) in a virtual environment. For example, a user could illustrate a psychological aspect of the experience as suffocating or constricting, and a haptic vest could be worn by the user and create (safe) physical stimuli for the user in a manner that reflects the illustrated aspect of the experience. During the process of affecting the experience, the stimuli (e.g. constriction) could lessen to match a visual representation of the suffocating or constricting psychological aspect of the experience being mitigated or eliminated. Tactile/haptic devices can also be powerful tools in inducing an out-of-body experience.

As an additional example, sensory devices can include a thermal device that allows for heating/cooling. Similar to haptic devices, the heating/cooling devices are used to enhance representations of digital models. For example, a cooling device can assist in the corporealization of an experience where a burning or intense aspect of the experience is cooled down. Implementations could include cooling a perceived sense of anger or frustration associated with the experience or quenching an intense psychological aspect of the experience to a less intense state by providing a cooling sensation through the thermal sensory device.

As an additional example, a user could associate a sense of doom or coldness with an aspect of her experience. The thermal device can act to corporealize (or supplement the corporealization of) this aspect of the experience by instantiating a cool state in the sensory device to correspond with the chilly feelings associated with the experience followed by warming the device in association with affecting the cold emotions.

Embodiments of XRNT can additionally, or alternatively, include a sensory device for propagating auditory signals (e.g., standalone speakers, headphones, etc.), olfactory signals, and/or gustatory signals. Olfactory signals can be delivered using an apparatus as known in the art that produces or releases fragrances or smells. For example, an olfactory device may release a relaxing set of fragrances that allow the user to more easily enter a meditative or calmed state. This, alone, may increase the user's ability to affect a corporealized experience. In addition, smell is known to be a powerful trigger for memories, and thus, an olfactory device can become an important anchor or trigger for affecting a corporealized experience, such as by using a pre-selected set of defined scents or aromas.

Olfactory devices can be especially relevant when affecting psychological aspects of an experience. A smell can be used within the olfactory device that elicits a powerfully positive or uplifting memory, and this memory can be used to help break trained behaviors (e.g., catastrophizing experiences, habitually imposing negative emotions on an experience, or similar) or to motivate the user to change aspects of the experience. For example, a user can be presented with a visual/digital representation of an aspect of the experience, which includes an unwanted psychological aspect. The disclose systems can, via an olfactory device, release a smell that triggers in the user a positive memory followed by a visual reduction of the psychological aspect of the experience or by a replacement of the psychological aspect of the experience with a pre-selected digital representation that elicits a positive effect in the user (e.g., makes the user happy). This can also be done, for example, while the user by interacts with a digital representation of the experience in a relieving action.

Olfactory devices can additionally, or alternatively, be used to train a user to feel certain ways. For example, a distinctive smell can be incorporated into a training session where the user is inundated with sensory signals that elicit a positive response from the user (e.g., empowers the user, makes the user happy, etc.). In some embodiments, the distinctive smell can be selected by the user. It may be beneficial to select a smell that does not elicit a powerful memory at the outset, as the user may be more prone to training with such a smell. Further, it should be appreciated that the distinctive smell can be any fragrance or smell or combination of fragrances.

In some embodiments, the distinctive smell is an aversive smell. An aversive smell can be used, for example, to break a user's learned behavior upon identification of triggers. For example, a user who catastrophizes an experience or causes a cascade of events leading to the unintentional onset of episodic pain (e.g., a user who misinterprets a stimulus as the beginning of a migraine and who through a series of psychological and/or physical acts causes the migraine to occur) can be trained with an aversive smell to recognize such behavior and/or to change such learned behavior. Because olfaction, particularly, can develop strong memories, a user may be able to use a portable vial of a fragrance associated with a trained behavior to initiate or catalyze positive behaviors. Such a feat would be made possible—and with a higher efficacy and in less time—through the use of the disclosed systems and methods.

In addition to, or alternatively from the sensory devices provided above, some embodiments of XRNT can include an intra-oral device, as known in the art, and/or a pre-selected set of defined taste substances (e.g., spices, confections, chemicals, etc.) to deliver gustatory signals to the user. When combined with the visualization of the user experience in the multidimensional sensory environment, these foregoing sensory devices can assist in corporealizing and affecting the user experience for the user's benefit.

As used herein, the term “user experience” is intended to describe a user's state or feelings. A user experience can include any of an emotional, psychological, physiological, and/or somatosensory experience, which when considered individually can constitute an experience in its own right (e.g., an emotional experience, a psychological experience, a physiological experience, and/or a somatosensory experience) or an aspect of the user experience (e.g., an emotional aspect of the user experience). In some instances, aspects of the user experience, such as a physiological experience, can include or associate with other aspects of the user experience; e.g., an emotional or psychological aspect of the physiological experience.

For example, fear is an emotion, and that emotion can be a “user experience.” As an additional example, depression can be a psychological “user experience,” but depression can have several emotional components, such as sadness and anger, each of which can form an “aspect” of the user experience that is depression. Further exemplifying the user of the term “user experience,” the pain from an open wound can have a physiological component the nociceptive signals from the damaged tissue telling the brain there is damage—and one or more emotional/psychological aspects (e.g., fear or depression caused by the waves of pain). The physiological nociceptive pain experience can be a physiological aspect of the user experience. The fear or depression caused by or associated with the pain can be emotional/psychological aspects of the user experience. The physiological aspect of the nociceptive pain can be considered to be associated with the emotional and psychological aspects (and vice versa).

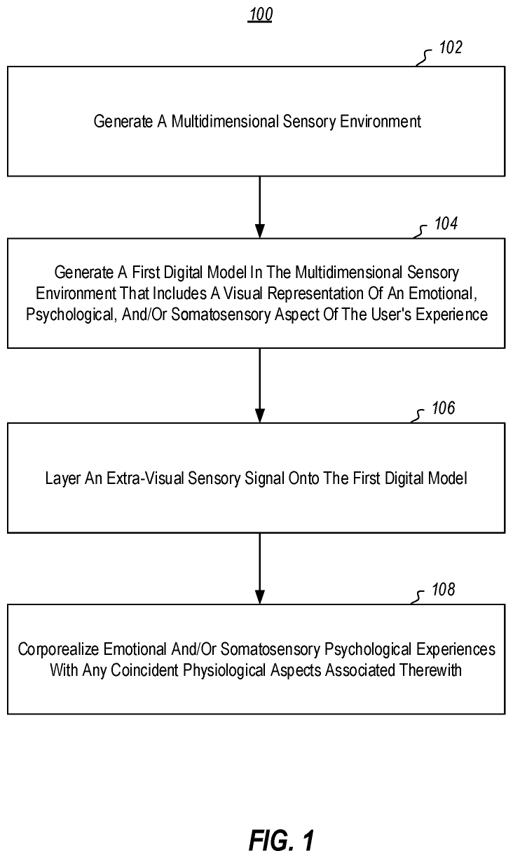

An example of a general computer-implemented method for corporealizing an individual's emotional or psychological experience, or the psychological component of a physiological experience or a somatosensory experience in an immersive environment is outlined in the method flow 100 of FIG. 1. For purposes of simplicity of explanation, the methodologies are shown and described as a series of blocks. However, it should be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described hereinafter.

As shown, the method 100 can include a step of generating a multidimensional sensory environment (act 102). A multidimensional sensory environment can be a digital environment having two or more spatial dimensions and capable of associating one or more extra-visual sensory signals to provide a user with a medium into which their experience can be corporealized. With specific reference to embodiments enabled by XRNT, particularly those utilizing an immersive technology such as VR, the multidimensional sensory environment can be a three-dimensional spatial environment that can be manipulated by the user as a “canvas” on which various visual aspects of their emotional, psychological, physiological, and/or somatosensory experience can be portrayed. In one embodiment, the multidimensional sensory environment can include a user-operated digital control panel for adding and manipulating digital models to the multidimensional sensory environment.

Through the control panel, the user can generate static and animated imagery and associate extra-visual sensory signals therewith. In some embodiments, the multidimensional sensory environment can include an avatar. The avatar can be a generic avatar, but in a preferred embodiment, the avatar reflects the user's likeness and/or image. The avatar can be useful for some individuals by providing anatomical reference points associated with the presentation of aspects of the experience in their own body. This can be beneficial, for example, in instances where the user's experience is associated with a somatosensory sensation. It may be difficult for the individual to verbalize the sensations, but through the multidimensional sensory environment, the individual can portray the sensations and communicate them more fully.

Accordingly, the method 100 of corporealizing the user's experience can additionally include generating a first digital model in the multidimensional sensory environment (act 104). The first digital model can include a visual representation of at least one of an emotional or psychological or somatosensory aspect of the user's experience. For example, a user attempting to corporealize her anxiety using XRNT can generate a first digital model in the multidimensional sensory environment that includes a visual representation of a constriction or weight around the avatar's chest. In reality, there is no actual constriction or weight around the user's chest, but to the user's perception of her experience, the digital model is accurate. In an alternative example, the user may choose to represent the anxiety with a dark black pulsating cloud that permeates through her avatar's chest and belly. The user can adjust the size, transparency, color, hue, intensity, or other visual aspects of the first digital model to more accurately reflect her own experience.

The method can further include layering an extra-visual sensory signal onto the first digital model (act 106). This can include, for example, associating an auditory, haptic, thermal, olfactory, and/or gustatory signal with the first digital model. Similar to the generation of the first digital model, the extra-visual sensory signal can be a digital representation of one aspect of the user's experience. Features associated with the extra-visual sensory signal can be adjusted so that characteristics relevant to that sense—e.g., location, frequency, intensity, depth, and/or overall impact of the signal—is conveyed in a manner and style that accurately reflects the user's experience. For example, in the running example of corporealizing a user's anxiety, the user can layer a haptic signal onto the visualized constriction or weight, causing a sensory device associated with the immersive technology to deliver the user-defined signal. This can include, for example, a haptic vest tightening (or vibrating to create the illusion of tightening). It should be appreciated that the user-defined (or computer-defined or helper-defined) extra-visual sensory signal can be implemented in various ways and in degrees of approximation to the user-defined signal and may be dependent upon the type of sensory device available. For example, a haptic vest may be an optimal mode for delivering the sensory signal but may not be available. In some instances, a handheld haptic element may act as a surrogate by delivering the user-defined intensity, or other defined aspect, of the tightening haptic vest through the handheld haptic element. That is, the handheld haptic element may vibrate or pulse in commensurate measure with the degree of tightening intended to be delivered by a haptic vest as an approximation of the intended extra-visual sensation.

It should be appreciated that act 106 can be repeated by layering additional extra-visual sensory signals onto the first digital model, whether of the same or different type as the initial layer. For example, the constricting anxiety can also be associated with a periodic thump that can be embodied by an additional haptic layer. A sound associated with the user's anxiety can also be layered onto the first digital model in addition to or separate from a coldness associated with the user's anxiety, which could be delivered through a speaker and thermal element, respectively, as described above.

It should also be appreciated that acts 104 and 106 can be repeated for additional digital models associated with emotional and psychological aspect of the user's experience in addition to related physiological and/or somatosensory aspects of the user's experience. By doing so, method 100 allows for complex emotional, psychological, and/or somatosensory experiences to be corporealized with any coincident physiological aspects associated therewith (act 108). In some embodiments, the corporealized experience may include a single, overarching psychological aspect associated with a plurality of physiologic stimuli. For example, a user experiencing chronic, systemic pain may associate a general sense of depression with the pain that isn't localized to any particular anatomic location. When visualized in a multidimensional sensory environment, the psychological aspect (e.g., the depression) may cover the entire avatar or be the background onto or the surroundings in which the avatar is displayed. It can also be represented as an image or animation or a combination of images or animations. For example, a sense of doom can be illustrated as a dark, looming figure or animal; additionally, or alternatively, the illustration of a dark, looming figure or animal can be accompanied by rolling cloud and intermittent flashes of lightning within the cloud.

The user's self-realized imagery reflecting the psychological aspects of the experience as it is perceived by the user can be additionally coupled with other sensory signals. Accordingly, the intensity of a psychological aspect of the experience can be mimicked in an aural signal—a heightened sense of anxiety accompanied by loud or booming thunderclaps or a lessened sense anxiety accompanied by low-frequency rumbles. Similarly, one or more haptic elements can be worn or held by the user that provide haptic signals according to a user's perception of the symptom. In the previous example of a heightened sense of anxiety, the loud or booming thunderclaps could be accompanied by an aggressive haptic feedback, shaking the user, whereas the low-frequency rumbles could be accompanied by a tremor within user-associated haptic elements.

The imagery and sensory signals associated with the psychological aspects of the experience can be selected from a pre-set list or illustrated by the user, the system, or a helper. In some embodiments, the user can describe the psychological aspect, and an associated computer system can render a digital model based on the description. The digital model can include instructions for sensory devices (e.g., sound level and type for aural signals, vibration frequency and duration for haptic signals, etc.).

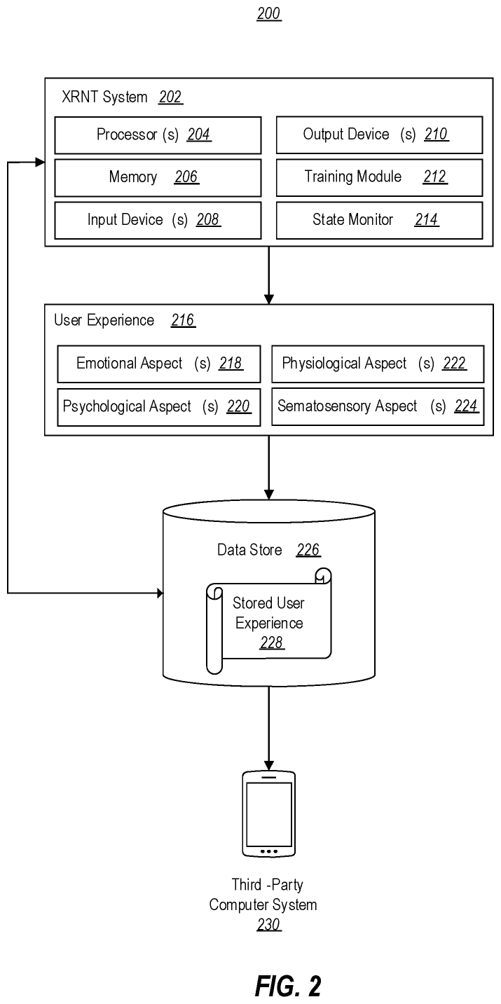

Turning now to FIG. 2, a computer architecture 200 is illustrated in which at least one embodiment described herein may be employed, such as the method 100 of FIG. 1. The computer architecture 200 includes a computer system, such as the XRNT system 202. The computer system includes at least one processor 204 and at least some system memory 206. The computer system may be any type of local or distributed computer system, including a cloud computer system and can additionally include modules for performing a variety of different functions. For instance, input devices 208 and output devices 210 can be configured to communicate with other computer systems or to communicate with a user. The input/output devices 208, 210 may include any wired or wireless communication means that can receive and/or transmit data to or from other computer systems and may be configured to interact with databases, mobile computing devices, embedded or other types of computer systems. Additionally, the input/output devices 208, 210 can include controllers and displays for communicating with a user. When using an XRNT system 202 utilizing an immersive technology, the input devices 208 can include paddles, joysticks, specialized pens, or even computer-recognized body movements (e.g., through a forward-facing camera mounted on an XR headset). The output devices 210 can include any display (e.g., 2D or immersive) and/or sensor device disclosed herein in addition to other output devices know in the art.

The computer system can additionally include a training module 212 that can communicate with input and/or output devices 208, 210 to enable various training modes to be executed on the computer system. The computer system can additionally include a state monitor 214 that is configured to monitor a user's physiological and/or psychological state, and in some embodiments communicates changes to the training module 212 for optimizing and/or personalizing training protocols. The state monitor 214 can, in some embodiments, communicate with biofeedback devices (e.g., personal health tracking watches and devices, transcutaneous electrical nerve stimulation (TENS) unit or similar devices) or data stores (such as data store 226) housing user-specific experience data 228.

The corporealized experience can be saved and/or shared with other individuals. Of note, any transferred user data may be encrypted and de-identified (i.e., anonymized) so that it conforms to patient privacy laws. Storing and/or sharing the corporealized experience can allow others, such as healthcare providers, loved ones, teammates, trainers, and coaches, an opportunity to more clearly understand an individual's experience, and that understanding and rich source of information can allow for precise treatments, augmented social behaviors and interactions, increased performance, and overall a more informed and individualistic approach to personal wellness and achievement than what is available with current systems and methods.

For example, including psychological and physiological aspects within the corporealized experience can enable a healthcare provider, in some instances, to prescribe a more effective treatment regimen. This can include a multi-disciplinary or multi-pronged treatment regimen that treats the physiological and psychological aspects of the condition. A physician may prescribe analgesics to treat physiologic aspects of the experience and refer the patient to a psychologist/psychiatrist for treatment of the psychological aspects of the patient's experience. Additionally, or alternatively, a patient can be prescribed a meditation routine or other stress-reducing activities (e.g., yoga, tai chi, qi gong, guided imagery, recreation, writing, exercise, breathing exercises, progressive muscle relaxation therapy, etc.).

In addition, in preferred embodiments, the caregiver or trainer can use the abilities of the system to create new, unique, or customized training to improve the psychological (as well as the physiological) health or performance of the individual. Although not necessarily required, the corporealized experience can be affected, such as within the context of XRNT.

The training feature of these systems can allow an individual to affect one or more aspects of their experience by, for example, confronting and/or exerting dominion over the corporeal representation, remediating the effect of the experience, allowing the user to identify and/or interrupt warning signs, cues, or triggers associated with an experience, and/or increase a user's performance—examples of which are provided below.

Embodiments of the present disclosure solve one or more problems in the art by enabling an individual to corporealize their experience, or aspects of their experience, and by doing so, make the experience tangible. This can have a great effect on the user, because the once ephemeral experience existing mostly as a plethora of indistinct, changing sensations is present before them in corporeal form where it can be confronted, addressed and/or controlled. In general, people feel more able to affect things they can see, but corporealizing the experience may also have the effect of demystifying the experience. Once the user can observe the experience, they can better understand its metes and bounds and how it can and should be controlled or affected. This behavior can be generally deemed a confrontation of the corporealized experience and can have a therapeutic benefit.

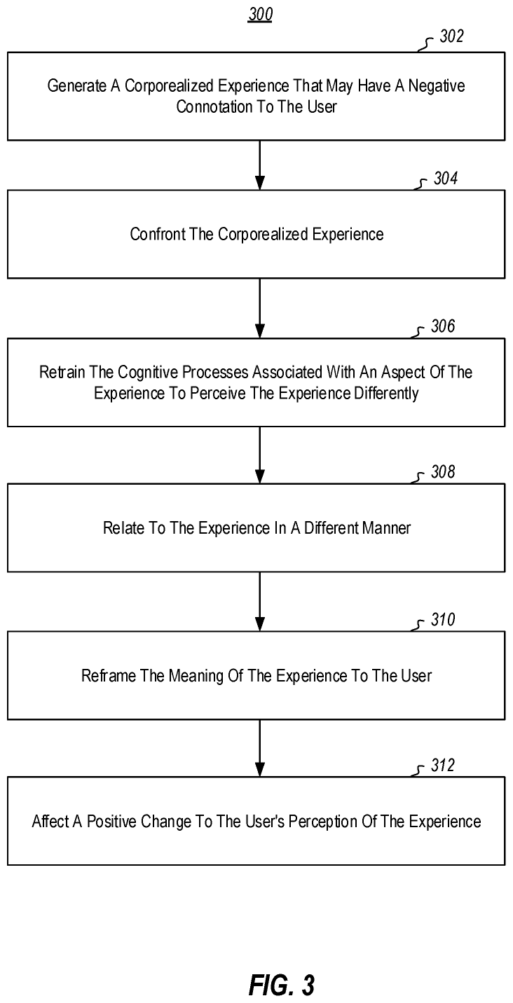

A method 300 for affecting an emotional and/or psychological experience, as illustrated in FIG. 3, can include generating a corporealized experience that may have a negative connotation to the user (act 302), and consequently, the user is likely to view the experience in a manner that the user perceives negatively (e.g., looming, dark, deep, abrasive, etc.). By confronting the corporealized experience (act 304), the user can control it. “Controlling” the experience can be accomplished in many ways and can include, for example, retraining the cognitive processes associated with an aspect of the experience to perceive the experience differently (act 306), relating to the experience in a different manner (act 308), and/or to reframing the meaning of the experience to the user (act 310). This can include modifying or morphing the visualized experience into a multi-sensory (e.g., audio/visual/tactile/olfactory) representation that elicits a different response from the user or that the user perceives positively (or neutrally). Thus, after training, when the experience presents itself outside of the multidimensional sensory environment, the user can control the experience and have improved performance/health (act 312).

In some embodiments, aspects of the experience are illustrated as images or animations that, during treatment, are modified or morphed into a digital model that elicits a positive response from the user. Referring to the example above of a psychological aspect being illustrated as a looming figure or animal, treatment of the psychological aspect embodied by the looming figure or animal can include modifying or morphing the looming figure or animal into a less ominous figure or animal. In one embodiment, the looming figure or animal is a dark hound, and during the treatment method, the dark hound is gradually illuminated or morphed into a cute puppy. As another example, a rolling thundercloud illustrating a psychological aspect of an experience can gradually slow and be broken or dissipated by a ray of sunshine. In another example, the throbbing cloud inside an avatar's chest and belly can gradually dissipate or morphed into a more positive somatosensory experience (e.g. glittering sparks representing the tingling sensations of a more positive nervous energy of excited anticipation). In these ways, embodiments of the present disclosure can enable users to break unhealthy or problematic associations with an experience or aspects of an experience by removing or breaking down negative psychological associations. In other embodiments the user can practice certain behaviors (e.g. breathing techniques or psychotherapeutic techniques) which help engender the transition to a more desirable experience or state. It should be appreciated that in some embodiments the digital model can be modified or morphed into a digital representation that is less oppressive or negative or to a representation that is neutral.

In some embodiments, the creation of the digital model of the experience or alleviation can be accompanied by audio-visual, other multimedia or multi-sensory guidance, such as a guided protocol or instructions, guided meditation, or affirmations. In some embodiments, the audio-visual guidance is used to reinforce a user's sense of empowerment to control the user's experience. In some embodiments, the multimedia guidance is used to train or reinforce techniques for handling aspects of the experience, such as cognitive behavioral therapy or specific instructions from a helper (e.g., physician, psychologist). The audio-visual guidance may comprise recordings of the user's own thoughts (e.g., self-affirmations of the patient's ability to control the experience). In some embodiments, the multi-sensory guidance may comprise an aural guidance and one or more other sensory guidances, such as a tactile or olfactory, to prompt or change.

In an exemplary embodiment, a user can use XRNT to corporealize a paralyzing fear associated with new social situations. The user can then spend time in the immersive environment viewing and understanding the corporealized form of her fear. Guided training provided by auditory sensory signals can assist the user in understanding, dissecting, questioning, reframing or changing the experience s, thereby removing some (or all) of its power to cause a perceived effect on the user in the real world. The user may still exhibit the fear in new social situations; however, he may now be able to recognize or even visualize/corporealize the various components of the experience, including any somatosensory components, and he will have more focused tools to engage with the various components of the experiences to maintain better control over the emotional state. For example, the user may start by drawing a large amorphous cloud to represent his fear. With guidance or self-exploration, the user may recognize that his fear, and thus the cloud, is actually comprised of several different emotions and somatosensory experiences. He may change the representation into a smaller cloud which represents negative emotions related to the fear and some sparks to represent tingling sensations of excitement. Indeed, it may be possible, for example, to reassess or morph a set of foreboding negative experiences (emotions and somatosensory experiences) into more positive experiences of tingling nervousness of excited anticipation.

In another embodiment, the corporealized experience, such as the foregoing paralyzing fear associated with new social situations, can be remediated using an XRNT system. The XRNT system can provide, in one instance, user-operated tools and/or preset paradigms within the immersive environment for helping the user to reduce one or more aspects of the experience (e.g., a size, intensity, shape, color, etc.). For example, the user can render a new digital model of the corporealized experience that represents one or more aspects being remediated. The user can be guided or self-taught within the multidimensional sensory environment how to transform the initially corporealized digital model of her experience into the new digital model of the attenuated experience. It should be appreciated that in addition to, or in lieu of, the visual aspects of the corporealized experience, any of the one or more layered sensory signals can be remediated and/or attenuated. In some embodiments, the system might even help by prompting or automatically making some of these changes based on accumulated data from the user or many users (anonymized) with similar fears.

As an exemplary implementation of the foregoing, a user may be struggling with anxiety but not understand the source or reason for it. The user can utilize an XRNT system (potentially as part of a comprehensive psychological treatment regimen) to enter an immersive environment that allows her to “draw” his emotions on an avatar. The user may select from multiple avatars, both human and non-human, to represent different aspects of her personality or different roles she plays in life (e.g. employee with a domineering boss, mother to a toddler with a chronic illness). The system allows the user to draw freeform shapes for various emotions, customizing color, size, shape, and various other aspects of the visual representation. The system could then also allow the user to associate a sound or other sensory signal with each specific emotion. The system may also prompt the user to think about somatosensory experiences associated with the emotion; for example, tightness in the gut, tension in the shoulders. The system may offer select prefabricated shapes and sounds (or other sensory signals) that the user may associate with those somatosensory sensations. In addition, they system may provide the user with a tactile device, such as a tactile suit or vest, which allows the user to simulate somatosensory sensations related to an emotion (e.g. tingling in the neck when fear comes on).

After this initial “drawing” process the system (or a helper) leads the user to examine each of the “drawn” items and to disambiguate the drawings further. In one embodiment, the user may discover an emotion, or the somatosensory reflection of that emotion, are really related to two different emotions or psychological states. The system may allow user to assign written/visual or audio labels to classify each. The user may also then be guided to disambiguate emotions based on their sources. For example, “this tension really comes up when my boss yells at me.” The system may allow the user to bring in metadata from her personal life (e.g., photos, videos) and link it to a context, such as an environment, or an emotion (e.g., photo of her sick child linked to the crushing weight in her chest which she associates with the sense of fear and helplessness).

In some embodiments, the system (or the helper) may also incorporate various coping and psychological therapies. For example, the user might be trained to practice relaxation breathing every time the heavy sensation in the pit of her stomach forms. Various other potential applications can be implemented as known in the art of psychological therapy.

In some embodiments, the XRNT system allows the user to separate distinct emotions or emotions related to different drivers into different avatars. The system may incorporate changes in the corporealized experience as the user practices therapies or (coping) strategies. For example, the representation of fear can start to dissolve or get smaller as the user practices a skill. The user might be able to interact with aspects of the corporealized experience in the virtual environment in various ways, such as by touching them, moving them, throwing them away, stepping out of the body that holds them (creating a sensation of leaving the issue behind or an out-of-body experience), washing, warming/cooling the experience, etc. Such changes could be enhanced through the various potential sensory devices, as well as by audio-visual stimuli.

In some embodiments, the user can identify an aspect of the corporealized experience that is similar (e.g., in one or more ways visually or in similarity to accompanying sensory signals) and that user identifies as a positive experience (or aspect of an experience). The user can reframe the corporealized experience having a negative connotation as being in the likeness of the positive experience. For example, the user may experience anxiety associated with a public speaking event, and that anxiety is corporealized in the immersive environment as a bright and erratically moving object about the user's avatar. The user, system, or helper can identify a corporealized form of excitement that is similar in one or more ways to the corporealized form of anxiety. For example, the user may experience excitement as a bright and erratically moving object, though different in some respects to the corporealized form of her anxiety. Through self-, computer-, or helper-guided instructions, the user can begin to recognize the similarities between the two emotions and reframe the anxiety as excitement. The system could then allow the user to actually practice morphing the experience inside themselves back and forth, until the user can morph one or more aspects of the experience more easily. This back and forth morphing could be paralleled through the corporealization in XRNT; in some cases, the corporealization leads and the user follows; in others the user could actually first try to morph within herself and then cause the corporealization to match her experience. Through the training process within the immersive environment, the user may be able to approach a public speaking event and when becoming anxious, identify at least the one aspect of her anxiety as a natural feeling of excitement.

Moreover, the systems described herein can also be used to actually affect or morph an (aspect of an) individual's experience in a positive way.

A particular application of XRNT can be applied in the treatment of Alexithymia. Alexithymia is a condition marked by an inability to identify or describe emotions. It is often associated with dysfunction in emotional awareness, social attachment, and interpersonal relating. The system similar to the example described above related to a user's anxiety can be used to help the person identify, describe, and then communicate emotions. For example, a young girl may be unable to communicate complex emotions. The child and her parent, potentially together with a therapist, use the XRNT system described herein to help the child begin to “draw” metaphorical representations of her emotions (e.g., a cloud of bees in her belly represents excitement, a loud horn sound for panic, an intense, audible vibration in a tactile vest to represent fear) on an avatar representing the child.

In doing so, the system enables the child, her parents, and/or therapist to establish an agreed upon multi-sensory vocabulary, which they can use during therapy sessions or other settings to communicate—and in a way that each party understands the emotion and/or psychological state being discussed.

The system, the child, the parents or therapist can also change these multi-sensory representations, allowing the child to explore under what circumstances they might have felt this different variant of the emotion. The child might even be able to change the environment or add other avatars or objects (e.g., by selecting prefabs in the system, by drawing them, or importing photos/videos) and then change the representation of their emotions based on the introduction of or changes in the environment, avatars or objects. This could be used to educate the child or even reveal previously unknown causes for emotions in the child.

In one aspect, the systems and methods disclosed herein can be used to train a subject to recognize certain emotional, physiological, psychological, and/or somatosensory cues associated with an experience—initially through corporealization in a multidimensional sensory environment—followed by training paradigms that teach an individual to cope with or prevent the progression of aspects of the experience. For example, subjects implementing one or more systems or methods can develop healthy behaviors to help cope with different levels or types of experiences, such as experiences like cascading (e.g. migraine headache cascade) or chronic pain events and, in some instances, symptoms of menopause.

Implementing the disclosed systems and methods, a subject can influence their own emotional or cognitive perception of the experience, reinforce positive outcomes, or even avoid future incidence of the experience, and make this behavior more likely with future episodes.

In some embodiments, the disclosed systems and methods can be adapted to identify and correct unwanted behaviors. For example, individuals who catastrophize an experience or focus on potentially unrelated physiological or psychological cues and thereby instigate or exacerbate an experience, can utilize the disclosed systems to identify and correct such unwanted behavior. In the example of catastrophizing an experience, the user can be trained to reduce or eliminate the amplification of psychological aspects and avoid future catastrophizing events. This can include, for example, visualizing separately the various emotional components and physiological components of the catastrophized experience and through active, passive, or responsive modes, learning to reduce or eliminate (the emotional or somatosensory) aspects of the experience to prevent or control current and/or future catastrophizing events. Such coping mechanisms can be learned more quickly by implementing one or more feedback devices described above, although in some embodiments, the visual feedback offered by the multidimensional sensory experience is sufficient to enable the user to learn control of or how to cope with catastrophized aspects of the experience.

Similarly, the disclosed systems can be used to identify and/or correct unwanted experiences having a psychological component. For example, a user could identify a bad habit that they want to break such as biting their fingernails. The user's desire to bite her fingernails can be visualized within the multidimensional sensory environment and physiological/somatosensory (e.g. tingling on the lips and teeth) or psychological cues that instigate or aggravate this desire can additionally be visualized. The treatment protocol can be activated, causing the digital models representing aspects of the bad habit to be reduced, eliminated, or modified, as described above. In some embodiments, instead of modifying the digital model to represent an image, animation, or other stimulus that is pleasing or representation that otherwise elicits a positive response within the user, the digital model is modified to represent an unpleasant stimulus or a representation that otherwise elicits an aversive response within the user. Over time or with sufficient feedback, the user can be trained to break the bad habit.

In a similar use case, the disclosed system and methods can be used to interrupt or stop the physiological and psychological experiences driving addiction. For example, a user could create a digital model of the emotional, psychological, physiological, and/or somatosensory states representing the onset of cravings. The system could then be used to teach the user to identify and diffuse or reframe the experiences to diffuse or reframe those experiences or somatosensory triggers and avoid actions related to his addiction—e.g., before lighting a cigarette or before eating another piece of chocolate.

In another example, the disclosed system and method can be used to interrupt or stop physiological and associated psychological experiences which lead to a negative physical or psychological event or condition (e.g., cascade preceding a migraine headache attack, build-up of anger leading to an outburst in a person with anger management issues). For example, patients with migraine headaches often experience a series of physiological and psychological experiences long before their pain starts. A migraine patient could create a digital model representing these physiological and emotional experiences and use the system to train her brain to recognize and interrupt or stop these systems before the pain ever starts. As an additional example, a menopausal woman could be taught to recognize and diffuse early symptoms of a menopause episode, like hot flashes, thereby interrupting or avoiding an emotional/psychological and/or somatosensory cascading into a more serious episode.

As an additional example, the disclosed systems and methods can be used to correct unwanted experiences, particularly unwanted experiences having a negative or shameful connotation, such as overeating. The user can visualize various different aspects of the experience in the multidimensional sensory environment, particularly various psychological aspects of the experience (e.g., shame, sadness, or disgust) or stimuli perceived by the user to be associated with unwanted behaviors and activate treatment protocols to help the user learn to cope with/release these psychological components, thereby also better controlling the unwanted behaviors.

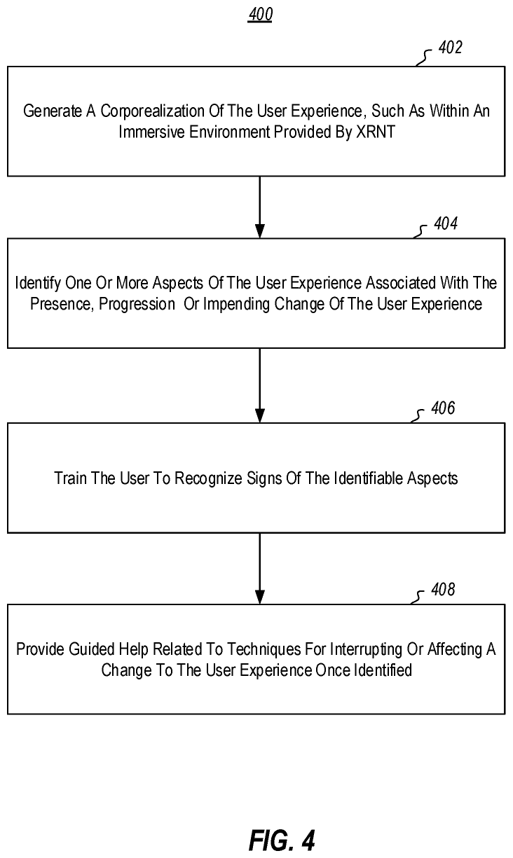

Method 400 of FIG. 4 illustrates a generalized paradigm for using the disclosed systems for corporealizing an emotional and/or psychological experience (which may be further influenced or associated with physiological and/or somatosensory sensations) to identify and sometimes interrupt warning signs, cues, triggers, or cascades associated with an experience. The method can include generating a corporealization of the user experience, such as within an immersive environment provided by XRNT (act 402). In some instances, the act of generating the corporealization of the user experience includes generating a sequence of corporealized experiences that together make up the user experience or that illustrate a sequence of experiences that result in the user experience. The method can additionally include identifying one or more identifiable aspects of the user experience associated with the presence, progression or impending change of the user experience (act 404). Based on the identifiable aspects, method 400 can additionally include training the user to recognize signs of the identifiable aspects (act 406) and provide guided help related to techniques for interrupting or affecting a change to the user experience once identified (act 408). It should be appreciated that in some embodiments, once the identifiable aspects of the user experience are recognized, one or more techniques for interrupting and/or affecting a change to the user experience can include confronting, controlling, and/or remediating the experience (as discussed herein).

As described above, emotions are a crucial component of human experience and emotional or psychological components are related to a large number of mental and physical states or experiences. Emotion can dramatically influence the perception of—and the experience of—a physical state. Accordingly, describing the emotional or psychological component and the physical or physiological component of a state or experience separately can be extraordinarily powerful. Treating the emotional component and physical component distinctly can lead to powerful therapies for improving performance.

For example, systems and methods disclosed herein can be implemented to increase the physical performance of an athlete. Athletes' performance can be influenced, and even hindered, by emotional or psychological aspects. In an exemplary case, endurance athletes commonly experience a phenomenon colloquially referred to as “hitting the wall.” This condition is marked by sudden fatigue, a perceived loss of energy, and a desire to cease the endurance activity. In some instances, “hitting the wall” is a psychological/emotional catastrophizing of physiologic symptoms such as depleted glycogen stores in the muscles and liver, as well as other potentially compounding factors. This can be a cue that the athlete has not properly regulated their caloric intake and that they should stop the activity and restore their energy supply; however, it is often also the case that the athlete has, in fact, properly regulated their caloric intake and has sufficient energy reserves. The experience of “hitting the wall” then reflects a combination of misinterpretation of physiological signals and an emotional catastrophizing of the emotional anguish and perceived pain. “Hitting the wall” may also be due to an inability to deal with (or lack of tools for dealing with) the emotional aspects (fear, anxiety, anguish) of pushing through a prolonged period of discomfort. How the athlete copes with these feelings and pushes through the wall can dramatically influence their overall performance.

An athlete can use the systems disclosed herein to create a digital representation of a mental experience that is impacting their performance, such as “hitting the wall” or the anguish associated with prolonged discomfort, and once visualized in a multidimensional sensory experience, the athlete can be trained to control the experience—thereby improving their performance.

The athlete can also be taught to more correctly (beneficially) interpret the physiological experiences and/or they can be taught to associate different emotions with the physiological experiences. In the exemplary case of an endurance athlete, a host of compounding factors such as induced chronic dehydration (e.g., glycogen binding water necessary for energy metabolism), muscle fiber breakdown driven by increased branched amino acid metabolism, and micro traumas due to the weight bearing, impact nature of the endurance activity can affect the athlete's psychological state and consequently the athlete's ability to maintain their pace. The experience to be corporealized and/or the experience to be immersed within using the disclosed systems and methods can include a combination of the athlete's physiological and psychological response to a perception of insufficient strength, endurance, and energy supplies to maintain a desired pace.

The systems disclosed herein beneficially enable users to control experiences or aspects of the experience to achieve improved performance and/or improved health—and are an improved way of doing so over what has previously been available. By providing the user with the tools and the ability to visualize (or corporealize in one or more sensory aspects) their individual physiological and psychological perception of an experience, the experience is embodied, and once embodied, its modification can permanently alter the user's perception of the experience, reframe its meaning, or retrain the user's brain to perceive or control the experience differently. Such training and/or treatment of experiences is more efficiently enabled by the multidimensional sensory experiences created by the disclosed systems and can more quickly or effectively cause improvements in the user's performance.

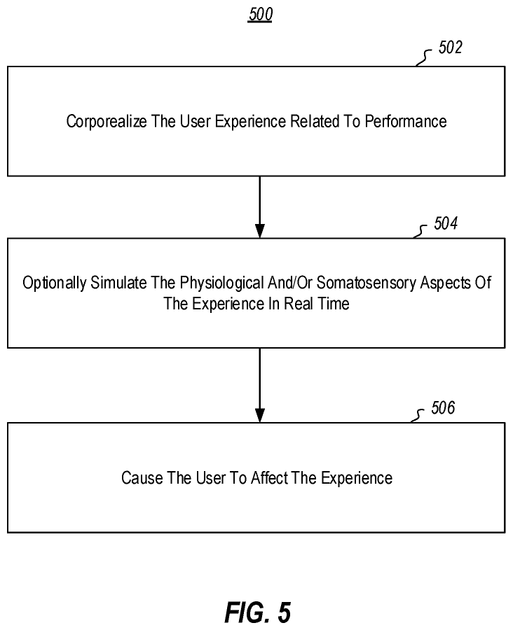

FIG. 5 illustrates a method 500 for affecting improved performance using a corporealized experience. In the running example of athlete performance, method 500 can include corporealizing the user experience related to performance (act 502), optionally simulating the physiological and/or somatosensory aspects of the experience in real time (act 504), and/or cause the user to affect the experience (act 506). Alternatively, the user can visualize and/or affect the experience while not concomitantly simulating the physical aspects of the experience. For example, an endurance athlete can be placed in a controlled environment (e.g., on a treadmill) and can engage the multidimensional sensory experience to visualize and/or treat “hitting the wall” when the athlete in reality “hits the wall.”

Alternatively, the athlete can create a multidimensional sensory experience that visualizes “hitting the wall” when the athlete is not in reality experiencing that experience and can engage in treatment methods while not currently experiencing the experiences. As above, this may prove advantageous for reframing future symptomatic experiences.

In some embodiments, the user experience is a positive one, such as being “in the zone,” a state of hyper-focus and apparently effortless performance. In such situations, the systems of the present disclosure can additionally be used to corporealize this positive experience and train the individual how to recognize aspects of the experience and to enter the experience more easily or more often or for longer periods of time—thereby increasing the performance of the individual.

In some embodiments, the system allows the user to control various aspects of the system utilizing biofeedback. For example, the user can specify where to collect biofeedback, how often and for how long biofeedback is to be collected, what types of biofeedback is to be collected, how the biofeedback is to be used or presented, etc. In some embodiments, the system can infer a user's health condition and/or ask the user to provide direct feedback regarding an emotional, psychological, physiological, and/or somatosensory sensation. The feedback can be solicited before, during, and after use of the disclosed systems. In some embodiments, a user can provide feedback upon the system request, or whenever the user wishes. In some embodiments, the feedback is not supplied by the user, but is automatically collected before, during, or after use of the system by examination of all or part of the user's body. Furthermore, as discussed above, the system can enable a user to visualize or otherwise sense the collected biofeedback directly and/or use it to adjust the corporealized experience or the training related thereto.

An example of the use of heart rate biofeedback is as follows. Along with the representation of the user's pain, the system provides a representation of the user's heart rate. As the user feels pain or focuses on antagonizing psychological aspects of the pain, her heart rate can rise. Lowering the user's heart rate or returning it to an optimal operational state (e.g., when exercising) may help the user relax or focus, and in some cases, this leads to a reduction in one or more aspects of the experience and can particularly reduce the intensity of psychological aspects of the user's experience. When the user manages to lower her heart rate into a target range, aspects of the emotional and/or psychological experience (and/or physiological and/or somatosensory sensations) can improve—e.g., decline, dissipate, or “heal.” In other words, the system can incorporate biofeedback techniques to provide the user with a way to drive treatment and obtain physical evidence of body condition improvement, while at the same time giving the user psychological training to help the user reduce or control aspects of their experience.

In the context of athletes described above, corporealizing experiences and learning to control aspects of the experience in a multidimensional sensory environment, such as XRNT, can improve their performance. Endurance athletes can learn to control their response to “hitting the wall,” baseball players can visualize their hitting “slump” and learn to control aspects of their psychological response to the “slump” to improve performance, and athletes, generally, can improve their mental toughness (e.g., their ability to more quickly turn a negative experience into a positive one). However, the same principles can be applied to many competitive academic circles where performance on standardized tests can create a negative emotional or psychological experience that hinders an individual's potential. Similar to the embodiments described above, an academic can utilize the disclosed systems, preferably XRNT, to corporealize the emotional and/or psychological experience associated with taking standardized tests and learn to positively affect that experience—thereby increasing their performance scores. In some embodiments, the systems disclosed herein can provide digital training sessions to users in a simulated test environment where the individual can learn in a near-equivalent setting how to identify and affect the negative emotional and/or psychological experience associated with standardized test taking.

Some embodiments of the present disclosure can additionally allow the transmutation of an emotion into a different, often more circumstantially useful or productive emotion. For example, a navy seal can be provided with a training system where he learns how to transmute anger, fear, or hopelessness into other emotions depending on the situation. Anger could be transmuted into aggression in a hand-to-hand combat situation or into high-presence, positivity energy in a negotiation with noncombatants. Systems disclosed herein can additionally provide scenarios where the user focuses more on morphing the multi-sensory representation of his emotions than on the environment. Some embodiments could provide the user with a way to visualize morphing rapidly through various different psychological states, including their somatosensory (e.g. tingling in neck) and physical (e.g. breath rate, heart rate) correlates. This could then be used to help make the morphing automatic or subconscious. For example, the user corporealizes what various emotions feel like to him (e.g. he could draw his fear as a cloud inside an avatar), including the associated somatosensory experience (e.g. a choking feeling in his throat could be represented by a red clinching throbbing ring around the windpipe inside the avatar). The user can be trained in techniques for morphing from one emotional state to another; e.g. the cloud could be transmuted into a combination of tingling sparks representing excitement and a burning throbbing sensation in the neck representing aggression; the red ring around the windpipe could be dissolved. In this way, a negative, weak set of experiences could be morphed into a pro-combat set of experiences. In some embodiments, such literal image could be augmented by other sensory stimuli (e.g. a heating device that actually warms the neck). In other embodiments, the corporealizations in the avatar could be augmented or replaced by real-world images. In some embodiments, the system could “randomly” morph the multi-sensory representations of emotion (and their related somatosensory experiences) and the user is allotted a period of time to duplicate that state within themselves.

Such systems could incorporate various biosensors to help the user train the mind and body (e.g. heart rate, breath rate) to, for example, help reinforce or interrupt emotional morphing.

It should be appreciated that the disclosed systems and methods can be applied to other embodiments with similar results. As a non-limiting example, the systems disclosed herein can be applied for assisting individuals in overcoming or affecting fear of interpersonal situations or for prepping for an athletic event (e.g. boxer before a fight).

The systems described herein could also be used to help more than one user; e.g. resolve conflict, teach empathy. For example, a couple struggling to communicate in a relationship can utilize embodiments of the systems disclosed herein to allow them to share with their partner how their emotional state changes as a result of what their partner's behavior or demeanor. By corporealizing the states, each partner is able to communicate more effectively to the other, and in some instances, affect a change. In particular, both individuals can enter an immersive environment (e.g., VR, MR, etc.) where they are each represented by a human avatar. In some embodiments, a therapist can be added as an observer represented by a human or non-human neutral avatar. Scenes can be selected that help reproduce real-world environments, and the system or the helper can guide the couple through scenarios. The couple is asked to corporealize or “draw” the most important emotions being generated in themselves during those scenarios (and potentially the emotions they think are happening in their partner).

Through this process, embodiments of the present disclosure instantiate a series of guided exercises that assist the users in confronting, recognizing, and/or understanding their partner's (and their own) emotions through the use of corporealized experiences. This can additionally include embodiments where the users are guided through a series of exercises to recognize emotions in themselves as warning signals and thereby work to affect (e.g., change) behavior.

In some embodiments, the couple is guided through therapies for managing the emotion and decoupling their own emotional response from the reality of the situation. For example, the scene might be frozen and one user is allowed to walk around the set, their avatar left behind, allowing them a third-person perspective of the entire situation, including their own emotion and their partners emotions. They can then choose to adjust what their real emotional level (e.g. the amount of anger that should be represented in their avatar) should be. It should be appreciated that other forms of therapy can be implemented using the disclosed systems as understood by one having skill in the art of couple's therapy.

In some embodiments of the disclosed system can be configured to collect various data and perform analytics, machine learning or artificial intelligence processes. Such processes could be used to improve training, and/or create ways to establish phenotypes or even diagnose certain conditions (e.g. Alixthemia).

In this description and in the claims, the term “computer system” or “computing system” is defined broadly as including any device or system—or combination thereof—that includes at least one physical and tangible processor and a physical and tangible memory capable of having thereon computer-executable instructions that may be executed by a processor. By way of example, not limitation, the term “computer system” or “computing system,” as used herein is intended to include immersive technologies, personal computers, desktop computers, laptop computers, tablets, mobile electronic devices (e.g., smartphones), microprocessor-based or programmable consumer electronics, minicomputers, mainframe computers, multi-processor systems, network PCs, distributed computing systems, datacenters, message processors, routers, switches, and even devices that conventionally have not been considered a computing system, such as wearables (e.g., glasses).

The memory may take any form and may depend on the nature and form of the computing system. The memory can be physical system memory, which includes volatile memory, non-volatile memory, or some combination of the two. The term “memory” may also be used herein to refer to non-volatile mass storage such as physical storage media.

The computing system also has thereon multiple structures often referred to as an “executable component.” For instance, the memory of a computing system can include an executable component. The term “executable component” is the name for a structure that is well understood to one of ordinary skill in the art in the field of computing as being a structure that can be software, hardware, or a combination thereof.

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled—whether in a single stage or in multiple stages—so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms—whether expressed with or without a modifying clause—are also intended to be synonymous with the term “executable component” and thus also have a structure that is well understood by those of ordinary skill in the art of computing.

While not all computing systems require a user interface, in some embodiments a computing system includes a user interface for use in communicating information from/to a user. The user interface may include output mechanisms as well as input mechanisms. The principles described herein are not limited to the precise output mechanisms or input mechanisms as such will depend on the nature of the device. However, output mechanisms might include, for instance, speakers, displays, tactile output, projections, holograms, and so forth. Examples of input mechanisms might include, for instance, microphones, touchscreens, projections, holograms, cameras, keyboards, stylus, mouse, or other pointer input, sensors of any type, and so forth.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example—not limitation—embodiments disclosed or envisioned herein can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product.

Transmission media can include a network and/or data links that can be used to carry desired program code in the form of computer-executable instructions or data structures and that can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Those skilled in the art will further appreciate that a computing system may also contain communication channels that allow the computing system to communicate with other computing systems over, for example, a network. Accordingly, the methods described herein may be practiced in network computing environments with many types of computing systems and computing system configurations. The disclosed methods may also be practiced in distributed system environments where local and/or remote computing systems, which are linked through a network (either by hardwired data links, wireless data links, or by a combination of hardwired and wireless data links), both perform tasks. In a distributed system environment, the processing, memory, and/or storage capability may be distributed as well.

Those skilled in the art will also appreciate that the disclosed methods may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Accordingly, methods and systems are provided for corporealizing and affecting emotional and psychological experiences and complex experiences that additionally include physiological and/or somatosensory aspects.

The concepts and features described herein may be embodied in other specific forms without departing from their spirit or descriptive characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

1-57. (canceled)

58. A computer system, comprising:
one or more processors; and
one or more hardware storage devices having stored thereon computer-executable instructions that, when executed by the one or more processors, configure the computer system to perform at least the following:
generate, via an immersive technology coupled to the computer system, a multidimensional sensory environment;
create a first digital model in the multidimensional sensory environment that comprises a visual representation of an emotional, psychological, or somatosensory user experience or aspect of said user experience;
receive a description of an extra-visual sensory signal corresponding to one or more of an aural, haptic, thermal, olfactory, or gustatory signal associated with the first digital model;
layer the extra-visual sensory signal onto the first digital model such that the extra-visual sensory signal is configured to be produced by a sensory device associated with the computer system;
produce a corporealized form of the user experience or aspect of said user experience in the multidimensional sensory environment, wherein producing the corporealized form of the user experience or aspect of said user experience comprises:
displaying the visual representation of the first digital model in the multidimensional sensory environment via the immersive technology; and
producing the extra-visual sensory signal associated with the first digital model at the sensory device; and
instantiate a guided protocol comprising audio-visual, or other multimedia or multi-sensory guidance, to affect a change to the user experience or aspect of said user experience.

59. The computer system of claim 58, wherein the corporealized form of the user experience or aspect of said user experience comprises a visual representation of the first digital model displayed by a display system configured for XR content, and the extra-visual sensory signal comprises an aural signal provided by a speaker or a haptic signal provided by a haptic device.

60. The computer system of claim 58, wherein the guided protocol reinforces a user's sense of empowerment to control the user experience or reframes a meaning of one or more aspects of the user experience.

61. The computer system of claim 60, wherein the computer-executable instructions additionally configure the computer system to generate a second corporealized form of a different user experience, wherein one or more aspects of the second corporealized form are related to aspects of the corporealized form of the user experience, and wherein the guided protocol is configured to identify or inform the one or more aspects related between the corporealized form and the second corporealized form of the user experience, or to assist in or train the user in experiencing or causing a transition between the corporealized form and the second corporealized form.

62. The computer system of claim 58, wherein the computer-executable instructions further configure the computer system to identify one or more aspects of the user experience that comprise a warning sign, a cue, a trigger, a precursor or step for a presence, progression of, cascades in, or impending change in the user experience or behavior.

63. The computer system of claim 62, wherein the guided protocol provides guided help related to techniques for recognizing and/or affecting change to the user experience or behavior based on the identified one or more aspects of the user experience.

64. The computer system of claim 63, wherein the instantiated guided protocol is configured to train the user to recognize the identified one or more aspects of the user experience and to morph the identified one or more aspects of the user experience into a positive or neutral corporealized experience.

65. A computer system, comprising:
one or more processors; and
one or more hardware storage devices having stored thereon computer-executable instructions that, when executed by the one or more processors, configure the computer system to perform at least the following:
generate, via an immersive technology coupled to the computer system, a multidimensional sensory environment;
create a first digital model in the multidimensional sensory environment that comprises a visual representation of an emotional, psychological, or somatosensory user experience or aspect of said user experience;
receive a description of an extra-visual sensory signal corresponding to one or more of an aural, haptic, thermal, olfactory, or gustatory signal associated with the first digital model;
layer the extra-visual sensory signal onto the first digital model such that the extra-visual sensory signal is configured to be produced by a sensory device associated with the computer system;
produce a corporealized form of the user experience or aspect of said user experience in the multidimensional sensory environment, wherein producing the corporealized form of the user experience or aspect of said user experience comprises:
displaying the visual representation of the first digital model in the multidimensional sensory environment via the immersive technology; and
producing the extra-visual sensory signal associated with the first digital model at the sensory device; and
morph the corporealized form of the user experience or aspect of said user experience into a multi-sensory representation.

66. The computer system of claim 65, wherein the corporealized form of the user experience or aspect of said user experience comprises a visual representation of the first digital model displayed by a display system configured for XR content, and the extra-visual sensory signal comprises an aural signal provided by a speaker or a haptic signal provided by a haptic device.

67. The computer system of claim 65, wherein the computer-executable instructions further configure the computer system to create a second visual representation corresponding to the multi-sensory representation, and wherein morphing the corporealized form of the user experience or aspect of said user experience comprises transmuting the corporealized form of the user experience or aspect of said user experience into the second visual representation that is circumstantially useful or productive.

68. The computer system of claim 65, wherein morphing the corporealized form of the user experience or aspect of said user experience comprises identifying an aspect of the corporealized form of the user experience or aspect of said user experience that is similar to a positive or neutral user experience and reframing the corporealized form of the user experience or aspect of said user experience as the positive or neutral user experience.

69. The computer system of claim 68, wherein the computer-executable instructions further configure the computer system to instantiate a guided protocol comprising audio-visual, or other multimedia or multi-sensory guidance, for identifying the aspect of the corporealized form of the user experience or aspect of said user experience that is similar to the positive or neutral user experience.

70. The computer system of claim 65, wherein the computer-executable instructions further configure the computer system to identify one or more aspects of the user experience that comprise warning signs, cues, triggers, precursors or steps for a presence, progression of, cascades in, or impending change in the user experience or behavior.

71. The computer system of claim 70, wherein the computer-executable instructions further configure the computer system to instantiate a guided protocol comprising audio-visual, or other multimedia or multi-sensory guidance, to train the user to recognize the identified one or more aspects of the user experience and/or to provide guided help related to techniques for affecting a change to the user experience or behavior based on the identified one or more aspects of the user experience.

72. A computer system, comprising:
one or more processors; and
one or more hardware storage devices having stored thereon computer-executable instructions that, when executed by the one or more processors, configure the computer system to perform at least the following:
generate, via an immersive technology coupled to the computer system, a multidimensional sensory environment;
create a first digital model in the multidimensional sensory environment that comprises a visual representation of an emotional, psychological, or somatosensory user experience or aspect of said user experience;
receive a description of an extra-visual sensory signal corresponding to one or more of an aural, haptic, thermal, olfactory, or gustatory signal associated with the first digital model;
layer the extra-visual sensory signal onto the first digital model such that the extra-visual sensory signal is configured to be produced by a sensory device associated with the computer system;
create a second digital model in the multidimensional sensory environment that includes a second visual representation comprising a physiological user experience or aspect of said physiological user experience; and
produce a corporealized form of the user experience or aspect of said user experience in the multidimensional sensory environment, wherein producing the corporealized form of the user experience or aspect of said user experience comprises:
displaying the visual representation of the first digital model and the second visual representation of the second digital model in the multidimensional sensory environment via the immersive technology; and
producing the extra-visual sensory signal at the sensory device.

73. The computer system of claim 72, wherein the corporealized form of the user experience or aspect of said user experience comprises a visual representation of the first digital model displayed by a display system configured for XR content, and the extra-visual sensory signal comprises an aural signal provided by a speaker or a haptic signal provided by a haptic device.

74. The computer system of claim 72, wherein the computer-executable instructions further configure the computer system to:
receive a second description describing a second extra-visual sensory signal associated with the second digital model; and
layer the second extra-visual sensory signal onto the second digital model such that the second extra-visual sensory signal is configured to be produced by the sensory device,
wherein producing the corporealized form of the user experience or aspect of said user experience comprises producing the second extra-visual sensory signal at the sensory device.

75. The computer system of claim 72, wherein the computer-executable instructions further configure the computer system to change the first digital model to an updated first digital model, the updated first digital model comprising a reduction or elimination of one or more of the visual representation of the first digital model or the extra-visual sensory signal of the first digital model.

76. The computer system of claim 75, wherein the computer-executable instructions further configure the computer system to instantiate a guided protocol comprising audio-visual, or other multimedia or multi-sensory guidance, to affect a change from the first digital model to the updated first digital model.

77. The computer system of claim 72, wherein the computer-executable instructions further configure the computer system to:
identify one or more aspects of the user experience that comprise warning signs, cues, triggers, precursors or steps for a presence, progression of, cascades in, or impending change in the user experience or behavior; and
instantiate a guided protocol comprising audio-visual, or other multimedia or multi-sensory guidance, to train the user to recognize the identified one or more aspects of the user experience and/or to provide guided help related to techniques for affecting a change to the user experience or behavior based on the identified one or more aspects of the user experience.
